(19) United States
(12) Patent Application Publication
(10) Pub. No.: US 2021/0228621 A1
(43) Pub. Date: Jul. 29, 2021

(54) COMPOSITION FOR SKIN WOUNDS

(71) Applicants: SUMITOMO SEIKA CHEMICALS CO., LTD., Kako-gun, Hyogo (JP); KYOTO PREFECTURAL PUBLIC UNIVERSITY CORPORATION, Kyoto-shi, Kyoto (JP)

(72) Inventors: Tomohisa TAKAGI, Kyoto-shi, Kyoto (JP); Jun ASAI, Kyoto-shi, Kyoto (JP); Tetsuya OKAYAMA, Kyoto-shi, Kyoto (JP); Katsura MIURA, Kyoto-shi, Kyoto (JP); Shigeki SAKAUE, Kako-gun, Hyogo (JP)

(73) Assignees: SUMITOMO SEIKA CHEMICALS CO., LTD., Kako-gun, Hyogo (JP); KYOTO PREFECTURAL PUBLIC UNIVERSITY CORPORATION, Kyoto-shi, Kyoto (JP)

(21) Appl. No.: 16/972,332

(22) PCT Filed: May 16, 2019
(86) PCT No.: PCT/JP2019/019579

§ 371 Date: Dec. 4, 2020

(30) Foreign Application Priority Data
Jun. 8, 2018 (JP) 2018-110717

(51) Int. Cl. A61K 33/00; A61P 17/02; A61K 9/00; A61K 9/08; A61K 47/38; A61K 47/32

(52) U.S. Cl. CPC A61K 33/00; A61P 17/02; A61K 47/32; A61K 9/08; A61K 47/38; A61K 9/0014

(57) ABSTRACT

This invention provides a means capable of treating skin ulcers. More specifically, the invention provides a composition for preventing and/or treating skin wounds, the composition comprising carbon monoxide.

### TECHNICAL FIELD

The present invention relates to, for example, a composition for preventing and/or treating skin wounds.

### BACKGROUND ART

A wound or ulcer that is damage to skin tissue etc. is usually called a “wound.” Specific examples include erosion, cuts, abrasions, burns, skin ulcers, pressure ulcers, and the like.

In particular, skin ulcers and pressure ulcers are caused by being bedridden or having diabetes, arteriosclerosis obliterans, vasculitis, and the like. Since many of these conditions are prolonged, the number of patients will surely increase, which is a major social problem, with the advent of the super aging society.

However, skin ulcers and pressure ulcers are still intractable diseases.

In the current situation, a sufficient therapeutic effect on intractable skin ulcers, pressure ulcers, etc. often cannot be expected with conservative treatment using a preparation for external use for treating wounds, such as an agent combining sucrose and povidone-iodine (Patent Literature (PTL) 1). When the condition becomes severe, surgical treatment is required (e.g., skin transplantation). Therefore, the development of a novel preparation for external use for skin that exhibits a more excellent therapeutic effect has been in demand.

### CITATION LIST

### Patent Literature

PTL 1: JP2000-38342A

### Non-Patent Literature

NPL 1: Journal of investigative Dermatology, (2006) 1.26, 1159-1167

### SUMMARY OF INVENTION

### Technical Problem

The present inventors found that a CO aqueous solution obtained by dissolving a high concentration of CO in water is effective in preventing and/or treating inflammatory gastrointestinal diseases. Therefore, the inventors attempted to prevent and/or treat skin ulcers by applying the same aqueous solution containing a high concentration of CO to skin ulcers.

An object of the present invention is to provide a novel means capable of treating skin ulcers.

### Solution to Problem

The present inventors found the possibility that a CO-containing composition may be capable of treating skin ulcers. The inventors made further improvements and completed the present invention.

The present invention encompasses, for example, the subject matter described in the following items.

Item 1. A composition for preventing and/or treating skin wounds, the composition comprising carbon monoxide.
Item 2. A composition for preventing and/or treating skin wounds, the composition comprising carbon monoxide and a solvent.
Item 3. The composition for preventing and/or treating skin wounds according to Item 2, wherein the solvent is water.
Item 4. The composition for preventing and/or treating skin wounds according to Item 2, wherein the solvent is isopropyl myristate.
Item 5. The composition for preventing and/or treating skin wounds according to any one of Items 2 to 4, further comprising a thickener.
Item 6. The composition for preventing and/or treating skin wounds according to Item 5, wherein the thickener is at least one member selected from the group consisting of carbomers (carboxyvinyl polymers) and hydroxyalkyl cellulose.
Item 7. The composition according to any one of Items 1 to 6, wherein the carbon monoxide concentration is 500 μM or more.
Item 8. The composition according to any one of Items 1 to 7, wherein the composition for preventing and/or treating skin wounds is for external use.
Item 9. The composition according to any one of Items 1 to 8, wherein the skin wound is a skin ulcer.
Item A-1. A method for preventing and/or treating skin wounds, the method comprising applying a composition comprising carbon monoxide to a subject.
Item A-2. The method according to A-1, wherein the composition is a composition comprising carbon monoxide and a solvent.
Item A-3. The method according to Item A-2, wherein the solvent is water.
Item A-4. The method according to Item A-2, wherein the solvent is isopropyl myristate.
Item A-5. The method according to any one of Items A-2 to A-4, wherein the composition further comprises a thickener.
Item A-6. The method according to Item A-5, wherein the thickener is at least one member selected from the group consisting of carbomers (carboxyvinyl polymers) and hydroxyalkyl cellulose.
Item A-7. The method according to any one of Items A-1 to A-6, wherein the concentration of carbon monoxide contained in the composition is 500 μM or more.
Item A-8. The method according to any one of Items A-1 to A-7, the method comprising applying the composition comprising carbon monoxide to an affected area of the subject.
Item A-9. The method according to any one of Items A-1 to A-8, wherein the skin wound is a skin ulcer.
Item B-1. A composition comprising carbon monoxide, for use in the prevention and/or treatment of skin wounds.
Item B-2. The composition according to Item B-1, further comprising a solvent.
Item B-3. The composition according to Item B-2, wherein the solvent is water.
Item B-4. The composition according to Item B-2, wherein the solvent is isopropyl myristate.
Item B-5. The composition according to any one of Items B-2 to B-4, further comprising a thickener.
Item B-6. The composition according to Item B-5, wherein the thickener is at least one member selected from the group consisting of carbomers (carboxyvinyl polymers) and hydroxyalkyl cellulose.
Item B-7. The composition according to any one of Items B-1 to B-6, wherein the carbon monoxide concentration is 500 pH or more.
Item B-8. The composition according to any one of Items B-1 to B-7, wherein the use in the prevention and/or treatment of skin wound is external use in the prevention and/or treatment of skin wounds.
Item B-9. The composition according to any one of Items B-1 to B-8, wherein the skin wound is a skin ulcer.
Item C-1. Use of a composition comprising carbon monoxide for the manufacture of a medicament for prevention and/or treatment of skin wounds.
Item C-2. The use according to Item C-1, wherein the composition comprises carbon monoxide and a solvent.
Item C-3. The use according to Item C-2, wherein the solvent is water.
Item C-4. The use according to Item C-2, wherein the sol vent is isopropyl myristate.
Item C-5. The use according to any one of Items C-2 to C-4, wherein the composition further comprises a thickener.
Item C-6. The use according to Item C-5, wherein the thickener is at least one member selected from the group consisting of carbomers (carboxyvinyl polymers) and hydroxyalkyl cellulose.
Item C-7. The use according to any one of Items C-1 to C-6, wherein the concentration of carbon monoxide contained in the composition is 500 μM or more.
Item C-8. The use according to any one of Items C-1 to C-7, wherein the medicament is a medicament for external use.
Item C-9. The use according to any one of Items C-1 to C-8, wherein the skin wound is a skin ulcer.

### Advantageous Effects of Invention

The composition for preventing and/or treating skin wounds encompassed by the present invention exhibits an excellent preventive and/or therapeutic effect.

### BRIEF DESCRIPTION OF DRAWINGS

FIG. 1 shows the results of hematoxylin eosin (HE) staining showing recovery from ulcers when a CO-containing composition (solvent: water) was applied to the ulcer site of skin ulcer model mice.

FIG. 2 is a graph showing the percentage (%) of an ulcer recovery (wound closure) area when a C-containing composition (solvent: a carbomer aqueous solution) was applied to the ulcer site of skin ulcer model mice. The horizontal axis shows the number of days, and the vertical axis shows the percentage (%) of the ulcer recovery area with respect to the initial ulcer area.

FIG. 3 is a graph showing the percentage (%) of an ulcer recovery (wound closure) area when a CO-containing composition (solvent: a hydroxyethyl cellulose aqueous solution) was applied to the ulcer site of skin ulcer model mice. The horizontal axis shows the number of days, and the vertical axis shows the percentage (%) of the ulcer recovery area with respect to the initial ulcer area.

FIG. 4 is a graph showing the percentage (%) of an ulcer recovery (wound closure) area when a CO-containing composition (solvent: a carbomer aqueous solution, a hydroxyethyl cellulose aqueous solution, or isopropyl myristate) was applied to the ulcer site of skin ulcer model mice. The horizontal axis shows the number of days, and the vertical axis shows the percentage (%) of the ulcer recovery area with respect to the initial ulcer area.

### DESCRIPTION OF EMBODIMENTS

Below, each embodiment of the present invention is described in more detail.

The composition for preventing and/or treating skin ulcers encompassed by the present invention comprises carbon monoxide (CO), and preferably comprises carbon monoxide (CO) and a solvent. This composition may be referred to as “the composition of the present invention.”

The solvent contained in the composition of the present invention is not particularly limited as long as it can retain (preferably dissolve) CO. Examples include water, organic solvents, and liquid mixtures of these. For liquid mixtures, the combination of solvents is not particularly limited. Further, the liquid mixtures can be homogeneous or heterogeneous. Examples of organic solvents include saturated aliphatic hydrocarbons, fatty acids, vegetable oils, carboxylic acid esters, alcohols, ethers, ketones, glycols, fatty acid esters, and the like. Examples of fatty acid esters include esters of fatty acids containing 12 to 18 carbon atoms and alkyl alcohols containing 1 to 20 carbon atoms. The alkyl alcohol particularly preferably contains 1 to 6 carbon atoms from the viewpoint of economy, safety, etc. The fatty acids may be saturated fatty acids or unsaturated fatty acids (preferably containing 1, 2, or 3 unsaturated bonds). More specific examples include lauric acid, myristic acid, palmitic acid, stearic acid, oleic acid, and the like. The alkyl alcohol may be straight-chain or branched-chain alkyl alcohol. More specific examples include methanol, ethanol, n-propanol, isopropanol, n-butanol, isobutanol, n-pentanol, isopentanol, n-hexanol, isohexanol, and the like. Particularly preferable examples of fatty acid esters include isopropyl myristate, isopropyl palmitate, and the like. Specific examples of organic solvents other than fatty acid esters include diisopropyl adipate, acetone, benzyl benzoate, isopropanol, fennel oil, almond oil, ethanol, ethylene glycol, 2-ethyl-1,3-hexanediol, diethyl ether, octyldodecanol, olive oil, oleic acid, glycerin fatty acid ester, crotamiton, geraniol-modified alcohol, synthetic squalane, sesame oil, wheat germ oil, ethyl acetate, normal butyl acetate, safflower oil, safflower oil fatty acid, ethylene glycol salicylate, diisopropanolamine, diethylene glycol, diethylene glycol monobutyl ether, diethylene glycol monomethyl ether, cyclohexanone, perilla oil, dipropylene glycol, dimethylpolysiloxane, squalene, diethyl sebacate, sorbitan acid fatty acid ester, soybean oil, soybean lecithin, propylene carbonate, medium-chain fatty acid triglyceride, camellia oil, corn oil, dodecylbenzene, triacetin, sorbitan trioleate, glyceryl tricaprylate, trichloroethane, concentrated glycerin, peppermint oil, octaacetyl sucrose-modified alcohol, castor oil, 1,3-butylene glycol, propionic acid, propylene glycol, propylene glycol fatty acid ester, benzyl alcohol, polyoxyethylene oleyl ether, polyoxyethylene sorbitan monostearate, polysorbate, polyethylene glycol, ethanol, methanol, methyl isobutyl ketone, methyl ethyl ketone, cottonseed oil, α-monoisostearyl glyceryl ether, polyethylene glycol monooleate, sorbitan monolaurate, coconut oil, polyoxyethylene lauryl alcohol ether, peanut oil, liquid paraffin, and the like. In particular, the solvent is preferably water or isopropyl myristate from the viewpoint of economy and ability to retain CO.

The composition of the present invention may further comprise a thickener. Examples of thickeners include monosaccharides and derivatives thereof, polysaccharides and derivatives thereof, fatty acids, amino acids and derivatives thereof, sugar alcohols, fatty acid esters, saturated aliphatic hydrocarbons, aliphatic alcohols, inorganic compounds, and the like. Specific examples include xanthan gum, gum arabic, guar gum, carrageenan, gellan gum, agar, locust bean gum, carbomer (carboxyvinyl polymer), carboxymethyl cellulose, sodium carboxyethyl cellulose, shellac, methyl cellulose, hydroxyethyl cellulose, hydroxypropyl cellulose, hydroxypropylmethyl cellulose, polyvinylpyrrolidone, polyvinyl alcohol, acrylic acid-alkyl methacrylate copolymr, polyacrylic acid salt, sodium alginate, propylene glycol alginate, ethyl cellulose, sodium carboxymethyl cellulose, magnesium aluminum silicate, sodium chondroitin sulfate, α-cyclodextrin, concentrated glycerin, polyethylene glycol, liquid paraffin, carob bean gum, glucono-δ-lactone, light anhydrous silicic acid, squalane, stearyl alcohol, aluminum stearate, lanolin, cetyl alcohol, gelatin, dextrin, urea, Vaseline, palmitic acid, potato starch, sodium hyaluronate, hydroxyethyl methylcellulose, castor oil, 1,3-butylene glycol, propylene glycol, polysorbate, polyethylene glycol, sodium metaphosphate, starch syrup, beeswax, methyl vinyl ether-maleic anhydride copolymer, rosin, dextrin palmitate, dextrin myristate, inulin stearate, 12-hydroxystearic acid, 1,3:2,4-dibenzylidene-D-sorbitol, L-leucine derivatives, L-isoleucine derivatives, L-valine derivatives, N-lauroyl-L-glutamic acid-α, and the like. Carbomers and hydroxyethyl cellulose are particularly preferable from the viewpoint of economy, safety, etc. These thickeners may be used alone, or in a combination of two or more.

For example, when water is used as the solvent, and a carbomer is used as the thickener, the composition preferably comprises 0.05 to 30 mass % of carbomer.

Further, for example, when water is used as the solvent, and hydroxyethyl cellulose is used as the thickener, the a of hydroxyethyl composition preferably comprises 0.1 to 14 mass cellulose.

The composition of the present invention preferably has a viscosity of about 0.3 to 1000000 centipoise (cp), more preferably about 0.5 to 500000 cp, and even more preferably about 0.8 to 100000 cp. The viscosity is a value measured by using a Brookfield viscometer after allowing the composition to be measured to stand at 25° C. for 30 minutes.

The composition of the present invention that comprises water as a solvent and that further comprises a thickener preferably has a viscosity within the above range although there is no particular limitation to this. The type and amount of the thickener used are also not particularly limited. It is preferable to appropriately select and use a thickener so that the viscosity falls within this range of the viscosity.

The composition of the present invention is preferably a composition for external use. Further, the composition of the present invention is preferably a liquid composition. The liquid composition as used herein includes a viscous composition (viscous liquid composition).

The composition of the present invention preferably comprises CO in a concentration of 500 μM or more. The CO concentration may be, for example, 550 μM or more, 600 μM or more, 650 μM or more, 700 μM or more, 750 μM or more, 800 μM or more, 850 μM or more, 900 μM or more, 950 μM or more, 1000 μM or more, 1050 μM or more, 1100 μM or more, 1150 μM or more, 1200 μM or more, 1250 μM or more, 1300 μM or more, 1350 μM or more, 1400 μM or more, 1450 μM or more, or 1500 μM or more. From the viewpoint of effects, in particular, the CO concentration is preferably 1000 μM or more. The upper limit of the CO concentration is not particularly limited, and CO can be incorporated into the solvent until saturation is achieved. The upper limit is, for example, 10000 μM or less, 9500 μM or less, or 9000 μM or less.

To obtain such a composition with a high CO concentration, for example, simply bringing CO into contact with, or blowing CO into, a solvent under ordinary pressure is usually insufficient. To prepare such a composition with a high CO concentration, it is preferable, for example, to perform replacement of CO by adjusting the pressure to, for example, 2 atm (or more) by using a gas-tight syringe or the like in a sealed container in which CO is collected.

The composition of the present invention is preferably used for the prevention and/or treatment of skin ulcers. The cause of the skin ulcers is not particularly limited. Examples of the causes include external injury, burns, diabetes, radiation exposure, peripheral vascular lesions, such as arteriosclerosis and venous stasis, collagen diseases (e.g., rheumatism), and the like. The covering epithelial damage that is milder than an ulcer is usually called “erosion” when the damage remains microscopically in the epithelial mucosa and does not reach the lower layer although the epithelium is macroscopically deficient. In the present specification, the term “ulcer” is used in the sense of including erosion unless otherwise specified.

The composition of the present invention can be applied not only to humans but also to non-human mammals. Examples of the non-human mammals include mice, rats, cats, dogs, monkeys, cows, horses, and the like.

The composition of the present invention can be administered, for example, once or multiple times (for example, 2 or 3 times) per day. The dose can be set appropriately. For example, the amount of the composition is preferably about 0.1 to 10 cc/day. As described above, the composition of the present invention is preferably a composition for external use, and in this case, the administration is performed by, for example, applying the composition to an ulcer site. The composition of the present invention may also be, for example, a liquid formulation or a foam-like formulation (foam formulation). The enema-foam-like formulation can be applied to the skin by ejecting a mixture of a liquid composition and an injection gas from a spray nozzle. For ejection, in particular, it is often the case that the liquid composition and the injection gas are mixed in a spray bottle immediately before ejection. The preferable CO concentration of the composition of the present invention in the form of a foam formulation may be the CO concentration obtained after mixing the liquid composition and the injection gas. For example, the CO concentration is preferably 500 μM or more after the liquid composition and the injection gas are mixed. Further, CO may be contained in the liquid composition or the injection gas, or both.

The composition of the present invention may further comprise other components as long as the effect of the present invention is not impaired. For other such components, known components can be used. In particular, components known to be used in compositions for external use can be used. Examples include disintegrating agents, lubricants (anti-aggregation agents), fluidizing agents, pH-adjusting agents, tonicity agents, absorption promoters, coloring agents, flavoring agents, antioxidants, antibacterial agents, preservatives, and the like.

Examples of disintegrating agents include carmellose calcium, low-substituted hydroxypropylcellulose, carmellose, croscarmellose sodium, partially pregelatinized starch, dried starch, sodium carboxymethyl starch, crospovidone, polysorbate 80 (polyoxyethylene sorbitan oleate), and the like.

Examples of lubricants (anti-aggregation agents) include talc, magnesium stearate, calcium stearate, colloidal silica, stearic acid, hydrous silicon dioxide, synthetic magnesium silicate, fine-particle silicon oxide, starch, sodium lauryl sulfate, boric acid, magnesium oxide, waxes, hydrogenated oil, polyethylene glycol, sodium benzoate, and the like.

Examples of fluidizing agents include silicic acid anhydride and the like.

Examples of pH-adjusting agents include hydrochloric acid, sodium hydroxide, citric acid, anhydrous citric acid, sodium citrate, sodium citrate dihydrate, anhydrous sodium monohydrogen phosphate, anhydrous sodium dihydrogen phosphate, and the like.

Examples of tonicity agents include sodium chloride, glucose, D-mannitol, glycerin, and the like.

Examples of absorption promoters include quaternary ammonium base, sodium lauryl sulfate, and the like. The content of these other components in the composition can be appropriately determined according to their type.

In the present specification, the term “comprise” or “contain” includes the meanings of consisting essentially of and consisting of.

### EXAMPLES

The present invention is described in more detail below.

### 1. Preparation of CO-Containing Composition

### Method for Preparing H₂O+CO Sample

Pure water (2.5 mL) was placed in a vial. (3 mL, produced by Maruemu Corporation), and the vial was sealed with a rubber stopper and an aluminum seal. Next, a gas (N₂ balance) having an arbitrarily adjusted CO concentration was collected in a Tedlar bag (1 L, produced by AS ONE Corporation). The air in the gas phase in the vial was replaced with the gas in the Tedlar bag until pressure reached about 2 atm by using a gas-tight syringe (M-GAN500, produced by Ito Corporation). Subsequently, the resulting product was allowed to stand until CO in the gas phase was dissolved in the liquid phase to reach equilibrium.

Table 1 shows the CO concentrations of the obtained CO-containing compositions (solvent: water). The CO concentration of the compositions was analyzed by gas chromatography (GC-2014 FID produced by Shimadzu Corporation).

TABLE 1

| CO in gas phase in Tedlar bag (%) | | | | | |
|---|---|---|---|---|---|
| (theoretical value) | | 8 | 34 | 67 | 100 |
| CO concentration of liquid | Solvent | 100 | 500 | 1000 | 1500 |
| phase obtained by actual | (H₂O) | | | | |
| measurement (μM) | | | | | |

### Method for Preparing IPM+CO Sample

Isopropyl myristate (IPM: Wako first grade, produced by Fujifilm Wako Pure Chemical Corporation) (2.5 mL) was placed in a vial (3 mL, produced by Maruemu Corporation), and the vial was sealed with a rubber stopper and an aluminum seal. Next, a gas (N₂ balance) having an arbitrarily adjusted CO concentration was collected in a Tedlar bag (1 L, produced by AS ONE Corporation). The air in the gas phase in the vial was replaced with the gas in the Tedlar bag until pressure reached about 2 atm by using a gas-tight syringe (MS-GAN500, produced by Ito Corporation). Subsequently, the resulting product was allowed to stand until CO in the gas phase was dissolved in the liquid phase to reach equilibrium.

The CO concentration of the obtained compositions was analyzed by gas chromatography (GC-2014 FID produced by Shimadzu Corporation). When the CO (%) in the gas phase in the Tedlar bag was 18%, the CO concentration of IPM was 1500 μM. When the CO (%) in the gas phase in the Tedlar bag was 100%, the CO concentration of IPM was 9000 μM.

### Administration to Skin Ulcer Model Mice—Example 1

Following a known method (Asai et al., Journal of Investigative Dermatology (2006) 126, 1159-1167), skin ulcer model mice were prepared. Specifically, spontaneously diabetic male mice (db/db mice) were received at 7 weeks of age, their backs were shaved, and the mice were then preliminarily kept in individual cages until 8 weeks of age. Each mouse was intraperitoneally injected with a mixed solution of ketamine and ceracetal, and the back of the mouse was shaved again under anesthesia. Then, a biopsy punch was used to induce a full-thickness skin defect wound with a diameter of 8 mm, thus producing skin ulcer model mice. Below, the thus-produced skin ulcer model mice were used as model mice.

The CO-containing composition (solvent: water, containing CO in a concentration of 1500 μM) was applied to the ulcer site of the skin ulcer model mice. The ulcer site was covered with a film (hydrophilic polyurethane dressing material; IV3000 dressing, transparent dressing) (without suturing). Thereafter, an operation of washing the ulcer site with physiological saline and applying the composition was repeated every other day. As a control experiment, an experiment was conducted in the same manner as above, except that water was used instead of the CO-containing composition.

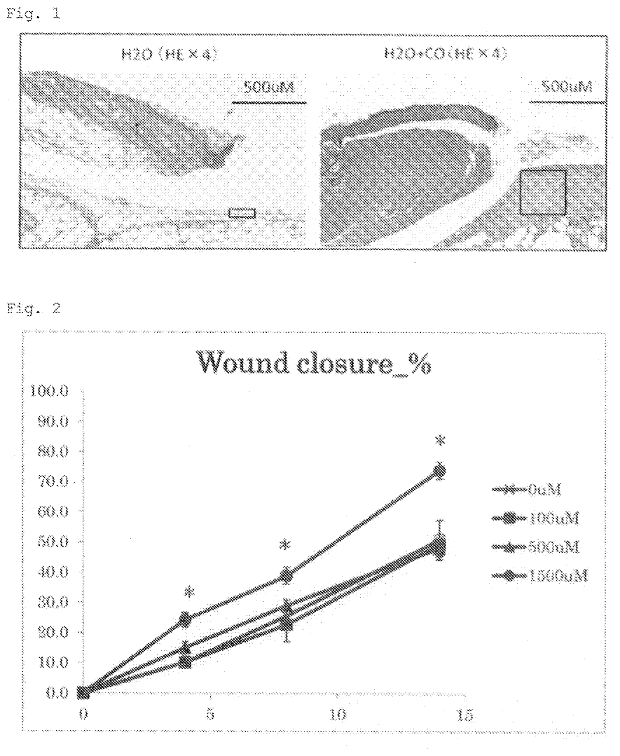

On day 14 from the application of the CO-containing composition, hematoxylin eosin (HE) staining of the ulcer sites was performed. FIG. 1 shows the results. The group to which the CO-containing composition was applied showed promoted formation of granulation tissue, compared to the group to which only water was applied.

### 2. Preparation of CO-Containing Composition

### Method for Preparing Carbomer+CO Sample

Pure water (300 g) was placed in a 500-mL poly beaker. While stirring was performed at 400 rpm with a three-one motor (BL-1200, produced by Shinto Scientific Co., Ltd.), 2.79 g of carbomer (Aqupec, 501E, produced by Sumitomo Seika Co., Ltd.) was added and dissolved by stirring at 240 rpm for 5 hours. Thereafter, 15.8 g of 6% sodium hydroxide aqueous solution was added and stirred for 30 minutes to prepare a carbomer aqueous solution. The carbomer aqueous solution (150 m) was placed in a 200-mL beaker, and the resulting product was allowed to stand in a constant temperature water tank at 25° C. for 30 minutes.

The carbomer aqueous solution (2.5 mL) was placed in a vial (3 mL, produced by Maruemu Corporation), and the vial was sealed with a rubber stopper and an aluminum seal. Next, a gas (N₂ balance) having an arbitrarily adjusted CO concentration was collected in a Tedlar bag (1 L, produced by AS ONE Corporation). The air in the gas phase in the vial was replaced with the gas in the Tedlar bag until pressure reached about 2 atm by using a gas-tight syringe (MS-GAN500, produced by Ito Corporation). Subsequently, the resulting product was allowed to stand until CO in the gas phase was dissolved in the liquid phase to reach equilibrium.

Table 2 shows the CO concentrations of the obtained CO-containing compositions. The CO concentration of the compositions was analyzed by gas chromatography (GC-2014 FID produced by Shimadzu Corporation).

TABLE 2

| CO in gas phase in Tedlar bag (%) | | | | |
|---|---|---|---|---|
| (theoretical value) | | 8 | 35 | 100 |
| CO concentration in liquid | Solvent (carbomer | 100 | 500 | 1500 |
| phase obtained by actual | aqueous solution) | | | |
| measurement (μM) | | | | |

### Method for Preparing Hydroxyethyl Cellulose+CO Sample

Pure water (250 g) was placed in a 500-mL poly beaker. While stirring was performed at 400 rpm with a three-one motor (BL-1200, produced by Shinto Scientific Co., Ltd.), 5.10 g of hydroxyethyl cellulose (CF-W, produced by Sumitomo Seika Chemicals Company, Limited) was added and dissolved by stirring at 240 rpm for 10 hours to prepare a hydroxyethyl cellulose aqueous solution. The hydroxyethyl cellulose aqueous solution (150 mL) was placed in a 200-mL beaker, and the resulting product was allowed to stand in a constant temperature water tank at 25° C. for 30 minutes.

The hydroxyethyl cellulose aqueous solution (2.5 mL) was placed in a vial (3 mL, produced by Maruemu Corporation), and the vial was sealed with a rubber stopper and an aluminum seal. Next, a gas (N₂ balance) having an arbitrarily adjusted CO concentration was collected in a Tedlar bag (1 L, produced by AS ONE Corporation). The air in the gas phase in the vial was replaced with the gas in the Tedlar bag until pressure reached about 2 atm by using a gas-tight syringe (MS-GAN500, produced by Ito Corporation). Subsequently, the resulting product was allowed to stand until CO in the gas phase was dissolved in the liquid phase to reach equilibrium.

The CO concentration of the obtained CO-containing compositions was analyzed by gas chromatography (GC-2014 FID produced by Shimadzu Corporation). When the CO (%) in the gas phase in the Tedlar bag was 100%, the CO concentration in the hydroxyethyl cellulose aqueous solution was 1500 μM.

### Administration to Skin Ulcer Model Mice—Example 2

The CO-containing composition that comprised carbomer prepared as described above was applied to the ulcer site of the skin ulcer model mice in the same manner as in Example 1. On days 4, 8, and 14, the area of the ulcer site (wound closure) was measured. As a control experiment, an experiment was conducted in the same manner as above, except that a composition that comprised only water and carbomer (the carbomer aqueous solution) was used instead of the CO-containing composition. FIG. 2 shows the results. The use of the composition that comprised only water and carbomer or the CO-containing composition that had a CO concentration of 100 μM showed almost no therapeutic effect on the skin ulcer. However, the use of the CO-containing composition that had a CO concentration of 500 μM showed a difference in the degree of recovery from the initial wound. Furthermore, the use of the CO-containing composition that had a CO concentration of 1500 μM showed a significant therapeutic effect on the skin ulcer. In FIG. 2, the asterisk (*) indicates a significant difference with respect to the group to which the composition comprising only water and carbomer was applied (P<0.05).

### Administration to Skin Ulcer Model Mice—Example 3

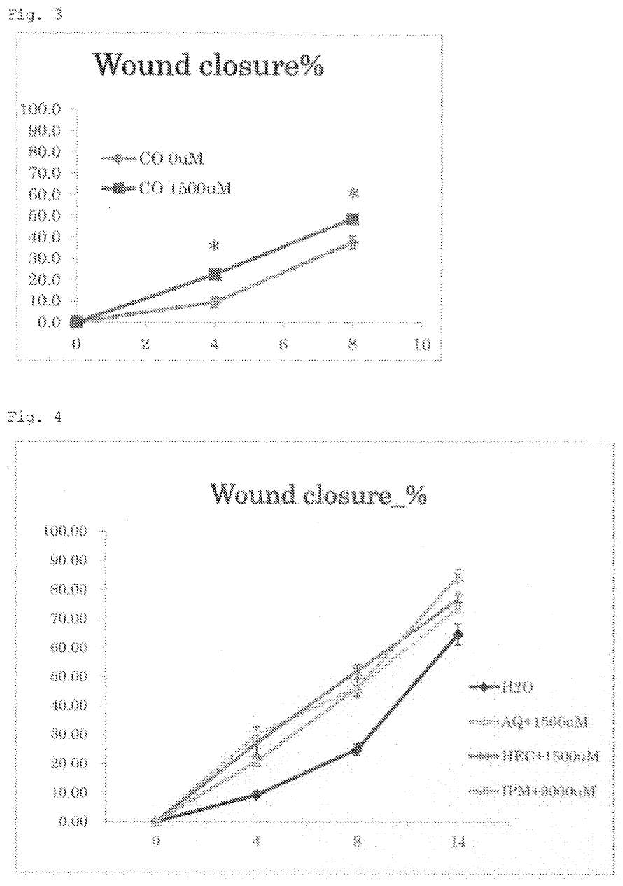

Further, analysis was performed using the skin ulcer model mice in the same manner as in Example 2, except that the CO-containing composition that comprised hydroxyethyl cellulose prepared as described above (CO concentration: 1500 μM) was used. FIG. 3 shows the results. The use of the CO-containing composition that had a CO concentration of 1500 μM showed a significant therapeutic effect on the skin ulcer. In FIG. 3, the asterisk (*) indicates a significant difference with respect to the group to which the composition that comprised only water and hydroxyethyl cellulose (the hydroxyethyl cellulose aqueous solution) was applied (P<0.05).

### Administration to Skin Ulcer Model Mice—Example 4

Analysis was performed using the skin ulcer model mice in the same manner as in Example 2, except that the CO-containing composition that comprised carbomer prepared as described above (solvent: water, CO concentration: 1500 μM), the CO-containing composition that comprised hydroxyethyl cellulose prepared as described above (solvent: water, CO concentration: 1500 μM), the CO-containing composition that comprised isopropyl myristate as the solvent (CO concentration: 9000 μM), or water was used. FIG. 4 shows the results. All of the CO-containing compositions showed a significant therapeutic effect on skin ulcer, compared to water. In FIG. 4, the CO-containing composition that comprised carbomer (solvent: water, CO concentration: 1500 μM), the CO-containing composition that comprised hydroxyethyl cellulose (solvent: water, CO concentration: 1500 μM), and the CO-containing composition that comprised isopropyl myristate as the solvent (CO concentration: 9000 μM) were denoted as “AQ+1500 μM,” “HEC+1500 μM,” and “IPM+9000 μM,” respectively.

1.-9. (canceled)

10. A method for preventing and/or treating skin wounds, the method comprising applying a composition comprising carbon monoxide to a subject.

11. The method according to claim 10, wherein the composition is a composition comprising carbon monoxide and a solvent.

12. The method according to claim 11, wherein the solvent is water.

13. The method according to claim 11, wherein the solvent is isopropyl myristate.

14. The method according to claim 11, wherein the composition further comprises a thickener.

15. The method according to claim 14, wherein the thickener is at least one member selected from the group consisting of carbomers (carboxyvinyl polymers) and hydroxyalkyl cellulose.

16. The method according to claim 10, wherein the concentration of carbon monoxide contained in the composition is 500 μM or more.

17. The method according to claim 10, the method comprising applying the composition comprising carbon monoxide to an affected area of the subject.

18. The method according to claim 10, wherein the skin wound is a skin ulcer.

19. The method according to claim 11,
wherein the solvent is water or isopropyl myristate, and
the concentration of carbon monoxide contained in the composition is 500 μM or more.

20. The method according to claim 19, wherein the composition further comprises a thickener.

21. The method according to claim 20, wherein the thickener is at least one member selected from the group consisting of carbomers (carboxyvinyl polymers) and hydroxyalkyl cellulose.
